(19) United States
(12) Patent Application Publication
(10) Pub. No.: US 2015/0107318 A1
(43) Pub. Date: Apr. 23, 2015

(54) METHOD OF PRODUCING ORGANIC PRODUCT FROM MOUNTAIN LITSEA

(71) Applicants: HENG-JUI HSU, New Taipei City (TW); TSU-YU HSU, New Taipei City (TW)

(72) Inventors: HENG-JUI HSU, New Taipei City (TW); TSU-YU HSU, New Taipei City (TW)

(21) Appl. No.: 14/060,565

(22) Filed: Oct. 22, 2013

(51) Int. Cl. A23L 1/223; C12P 17/06; C12P 7/02; C07K 14/78; C12N 1/16; C12G 3/02; C05F 17/00; C05F 5/00; C10L 8/00; A61K 8/97; C12N 9/00; A23L 1/30

(52) U.S. Cl. CPC A23L 1/223; C12N 9/00; C12P 17/06; C12P 7/02; C07K 14/78; A23L 1/3002; C12G 3/02; C05F 17/0027; C05F 5/00; C10L 8/00; A61K 8/97; C12N 1/16; A23V 2002/00
USPC 71/5; 435/41; 435/183; 435/125; 435/155; 435/71.1; 71/8; 435/255.1

(57) ABSTRACT

The method contains the following steps. In step A, supersonic waves are applied to Mountain Litsea fruits for excitation and sterilization. In step B, the inferior fruits and the cleaning water are removed from the water tank, placed in a fermentative tank, and left for fermentation. After the fermentation, a pressing and separation process is conducted. In step C, the superior fruits are placed in a separate fermentative tank for fermentation into mofetil alcohol. In step D, the superior fruits are alternatively mixed with pure water, crushed, and placed in a boiler for aging and sterilization. Then the crushed and aged fruits are placed in yet another fermentative tank with pure water for fermentation. After the fermentation, a pomace separation process is conducted. The separated pomace becomes a Mountain Litsea wine lees, whereas the separated water becomes a Mountain Litsea enzymatic liquid.

### TECHNICAL FIELD OF THE INVENTION

The present invention is generally related to Mountain Litsea, and more particular to a method of producing versatile organic products from Mountain Litsea fruits.

### DESCRIPTION OF THE PRIOR ART

Plants have the ability to synthesize a wide variety of chemical compounds that are used to perform important biological functions, and many of them have beneficial effects on long-term health when consumed by humans, and can be used to effectively treat human diseases. Mountain Litsea, or Fragrant Litsea, is one such plant.

Experiments show that the limonene content of Mountain Litsea can effectively suppress cough, eliminate sputum, and stop bacteria from reproducing.

The Nerol content of Mountain Litsea can also be used to produce essence with fruit fragrance, and essence for cosmetic purpose. The geraniol content of Mountain Litsea can also be used to produce various editable essences, vitamin A, mosquito repellent, antibacterial agent, cigarette ingredient, etc.

As describe, Mountain Litsea is a valuable plant but most people only uses it as seasoning. For example, R.O.C. Taiwan Patent No. M372723 teaches a device for producing Mountain Litsea oil for seasoning and skin care. The teaching can also produce a single kind of Mountain Litsea product, which is not versatile enough. In addition, the process requires the application of organic and inorganic solvents to dissolve Mountain Litsea and to produce artificial compounds. It fails to fully extract the rich elements of Mountain Litsea through a truly natural process.

### SUMMARY OF THE INVENTION

Therefore the present invention provides a healthy, non-toxic, zero-waste, non-pollutant, and versatile method of producing various organic products from the fruits of Mountain Litsea.

The method contains the following steps. In step A, supersonic waves are applied to Mountain Litsea fruits so as to excite the content of the fruits and to sterilize the fruit skins. In step B, the inferior ones among the Mountain Litsea fruits and the cleaning water are removed from the water tank, placed in a fermentative tank, and left for fermentation. After the inferior Mountain Litsea fruits ferment to an appropriate degree, a pressing and separation process is conducted. In step C, the superior ones among the Mountain Litsea fruits are placed in a separate fermentative tank for fermentation into mofetil alcohol. In step D, the superior ones among the Mountain Litsea fruits are alternatively mixed with an appropriate amount of pure water, crushed, and placed in a boiler for aging and sterilization. Then the crushed and aged Mountain Litsea fruits are placed in yet another fermentative tank with pure water for fermentation. After the fermentation reaches an appropriate degree, a pomace separation process is conducted. The separated pomace becomes a Mountain Litsea wine lees, whereas the separated water becomes a Mountain Litsea enzymatic liquid.

Compared to the prior arts, the present invention has the following advantages.

Firstly, the amino acid, protein, carbohydrate in Mountain Litsea are processed through purely naturally means to be fully extracted into various organic products.

Secondly, the products from the Mountain Litsea have built-in sterilization function for suppressing bacteria. Therefore no preservative or similar chemical is required.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become apparent to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a flow diagram showing the steps of the present invention.

FIG. 2 is a schematic diagram showing the products produced from a step B of the present invention.

FIG. 3 is a schematic diagram showing the products produced from a step C of the present invention.

FIG. 4 is a schematic diagram showing the products produced from a step D of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

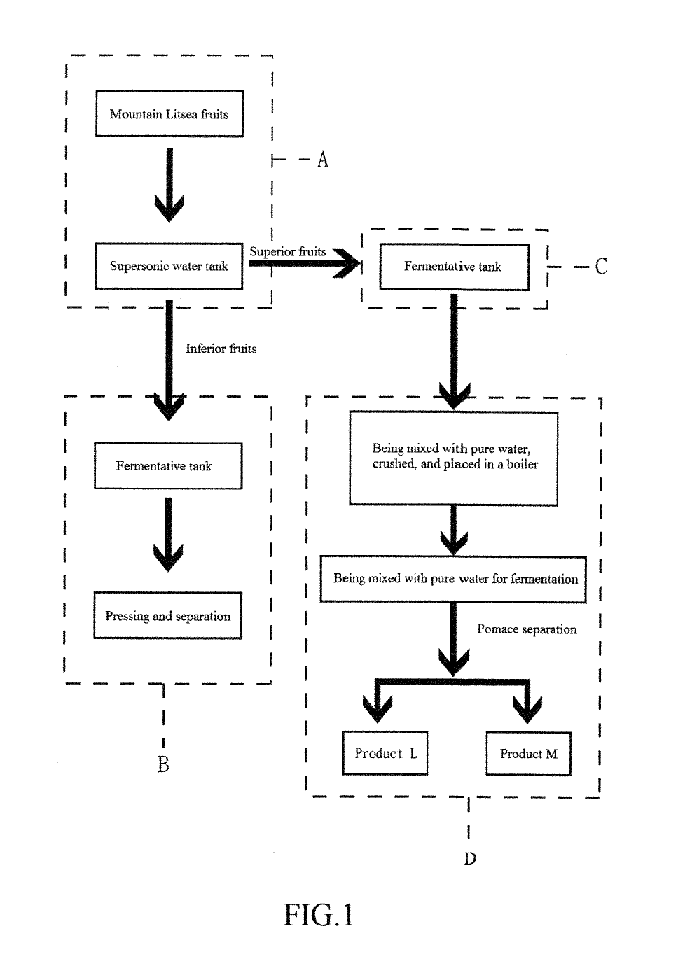

As shown in FIG. 1, the present invention contains the following steps. In step A, Mountain Litsea fruits are placed in a water tank and supersonic waves are applied to the Mountain Litsea fruits in the water tank so as to excite the content of the fruits and to sterilize the fruit skins. In step B, the inferior ones among the Mountain Litsea fruits are removed from the water tank, placed in a fermentative tank along with the water in the water tank, and left for fermentation. After the inferior Mountain Litsea fruits ferment to an appropriate degree, a pressing and separation process is conducted. In step C, the superior ones among the Mountain Litsea fruits are placed in a separate fermentative tank for fermentation until the fermentation reaches an appropriate degree. In step D, the superior ones among the Mountain Litsea fruits can also be mixed with an appropriate amount of pure water, crushed, and placed in a boiler for aging and sterilization. Then the aged and sterilized Mountain Litsea fruits are placed in yet another fermentative tank with pure water for fermentation. After the fermentation reaches an appropriate degree, a pomace separation process is conducted. The separated pomace becomes Mountain Litsea wine lees (i.e., a product L of the present invention), whereas the separated water becomes Mountain Litsea enzymatic liquid (i.e., a product M of the present invention).

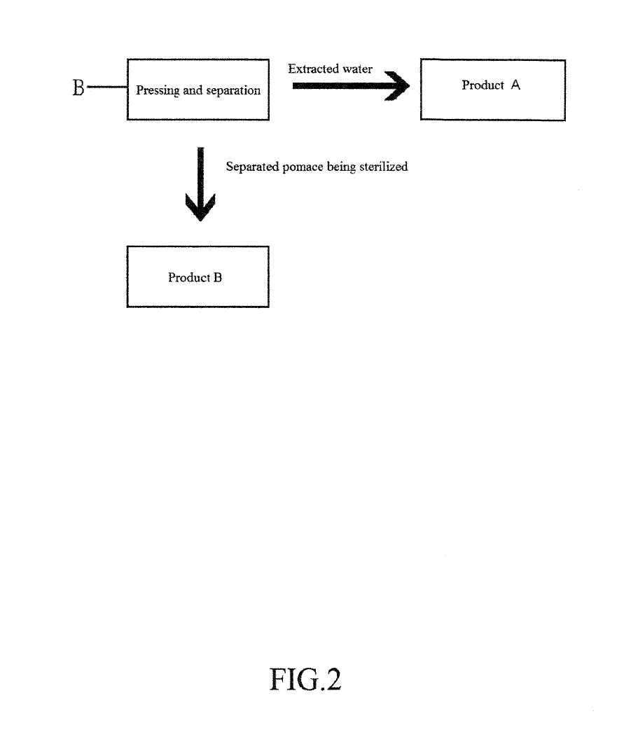

As shown in FIG. 2, in step B, the inferior Mountain Litsea fruits and dirty water ferment to an appropriate degree, and a pressing and separation device is applied. The extracted water can be used an enzymatic amino acid liquid fertilizer for agricultural purpose (i.e., a product A of the present invention). The separated pomace, after sterilization, can be used as a culture medium for fungus, mushroom, Ganoderma lucidum, etc.

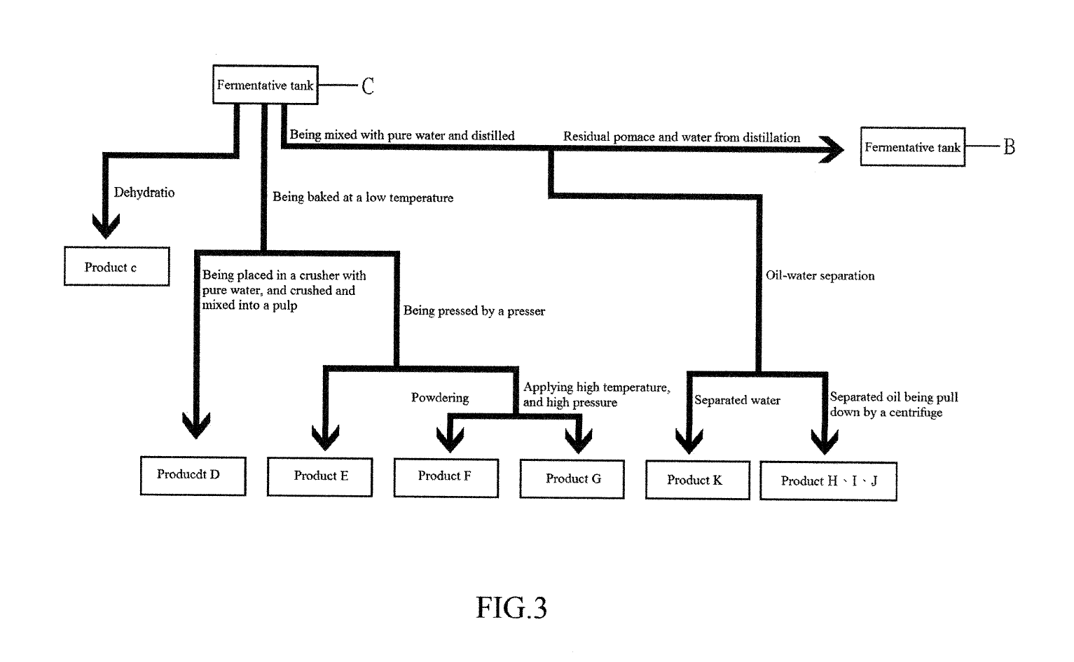

As shown in FIG. 3, in step C, the superior ones among the Mountain Litsea fruits are placed in a separate fermentative tank for half-dry natural fermentation to an appropriate degree into mofetil alcohol. Then, by a dehydration process through a warm or cool dryer, Mountain Litsea fruits that can be short-term or long-term stored using refrigerator or freezer are produced (i.e., a product C of the present invention). Alternatively, the superior Mountain Litsea fruits can be baked at a low temperature between 40 and 90° C. to enhance the release of mofetil alcohol, placed in a crusher with pure water, and crushed and mixed into a pulp (i.e., a product D of the present invention). Alternatively, the superior Mountain Litsea fruits can be pressed by a presser to produce Mountain Litsea oil (i.e., a product E of the present invention). The oil residue from the above pressing process can undergo a powdering process into Mountain Litsea powder (i.e., a product F of the present invention). Alternatively, the oil residue can undergo a high-temperature, high-pressure process, and becomes a highly valuable green fuel product (i.e., a product G of the present invention). In step C, the superior ones among the Mountain Litsea fruits are placed in a separate fermentative tank for half-dry, natural fermentation to an appropriate degree into mofetil alcohol. Then, alternatively, by adding pure water and conducting a distillation process at 100° C., oil and water are naturally separated after distillation. Through an oil-water separation device, the separated water is a pure dew (i.e., a product K of the present invention). The separated oil, then by a centrifuge's pull down, is further separated into an upper-layered oil (i.e., a product H of the present invention), a middle-layered oil (i.e., a product I of the present invention), and a bottom-layered collagen (i.e., a product J of the present invention).

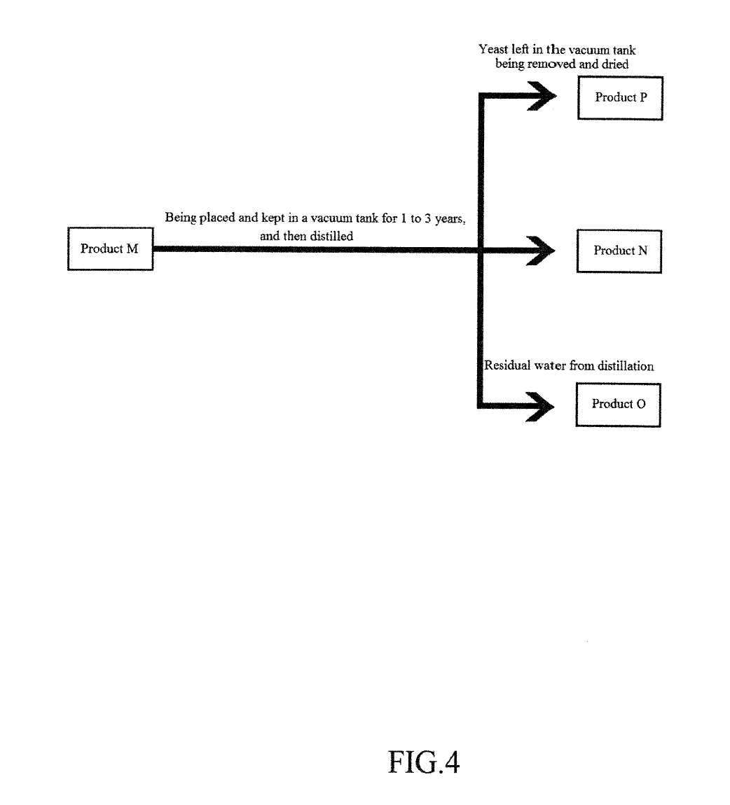

As shown in FIG. 4, the product M from the step D can be further processed. The product M can be placed and kept in a vacuum tank for 1 to 3 years, and then distilled to obtain Mountain Litsea wine (i.e., a product N of the present invention). In addition, the yeast left in the vacuum tank can be removed and dried into Mountain Litsea yeast (i.e., a product P of the present invention), and the residual water left from the distillation becomes Mountain Litsea mofetil alcohol anhydride fermented (i.e., a product O of the present invention).

The present invention has the following advantages. Firstly, the amino acid, protein, carbohydrate in Mountain Litsea are processed through purely naturally means to be fully extracted into various organic products. Secondly, the products from the Mountain Litsea have built-in sterilization function for suppressing bacteria. Therefore no preservative or similar chemical is required. Thirdly, the entire process is healthy, non-toxic, zero-waste, non-pollutant, and therefore is truly environmentally friendly.

The various organic products produced by the present invention are summarized as follows.

Product A: enzymatic amino acid liquid fertilizer suitable for various agricultural purposes.

Product B: suitable as a culture medium for fungus, mushroom, Ganoderma lucidum.

Product C, D: Mountain Litsea fruits or pulp that can be used for wine making, or along with bread, beverage, various meat products, vegetables, or used for food processing.

Product E: Mountain Litsea oil abundant with vitamin E capable of anti-oxidation, eliminating free agent, and slowing down aging, that can be used as nutritious and healthy food products, editable oils, and for adding in cosmetic and skincare products, and for refreshment, bacteria and mosquito suppressing,

Product F: Mountain Litsea powder that can replace pepper powder.

Product G: a green fuel product that can be used for industries, and as a strategic resource.

Product H: Mountain Litsea oil abundant with vitamin E capable of anti-oxidation, eliminating free agent, slowing down aging, soothing tension, and pain relieving, without side effect that can be used as cosmetic and skincare products.

Product I: Mountain Litsea oil abundant with vitamin E capable of anti-oxidation, eliminating free agent, and slowing down aging, that can be used for medical treatment, as nutritious and healthy food products, as cosmetic and skincare products, and as seasoning to add a unique flavor.

Product J: collagen that can be used as nutritious and healthy food products, and cosmetic and skincare products.

Product K: pure dew that can be used as nutritious and healthy food products, cosmetic and skincare products, and for replacing the water added in food preparation process (e.g., bread making) to add an unique flavor.

Product L: Mountain Litsea wine lees that can be for adding into and making dishes and cooking sauces.

Product M: Mountain Litsea enzymatic liquid that can be used as nutritious and healthy food products, and for adding into and making dishes and cooking sauces.

Product N: Mountain Litsea wine that can be used as nutritious and healthy food products and table wines.

Product O: Mountain Litsea mofetil alcohol anhydride fermented that can be used as nutritious and healthy food products, and for adding into and making dishes and cooking sauces.

Product P: Mountain Litsea yeast that can be used as nutritious and healthy food products, cosmetic and skincare products, and for adding into and making dishes and cooking sauces.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A method of producing organic products from Mountain Litsea fruits comprising the steps of:
A: placing a plurality of Mountain Litsea fruits with cleaning water in a water tank, and applying supersonic waves to the fruits to excite fruit content and to sterilize fruit skins;
B: selecting and placing the inferior ones from the Mountain Litsea fruits, together with the cleaning water, in a fermentative tank for fermentation, and conducting a pressing and separation process after the fermentation reaches an appropriate degree;
C: selecting and placing the superior ones from the Mountain Litsea fruits in a second fermentative tank for fermentation until the fermentation reaches an appropriate degree; and
D: selecting and crushing the superior ones from the Mountain Litsea fruits with pure water, aging and sterilizing the crushed fruits in a boiler, placing the aged and sterilized fruits in a third fermentative tank with pure water for fermentation, and conducting a pomace separation process after the fermentation reaches an appropriate degree, where the separated pomace is Mountain Litsea wine lees, and the separated water is Mountain Litsea enzymatic liquid.

2. The method according to claim 1, wherein the water separated by the pressing and separation process is an enzymatic amino acid liquid fertilizer for agricultural purpose.

3. The method according to claim 1, wherein the pomace separated by the pressing and separation process is further sterilized and used as a culture medium.

4. The method according to claim 1, further comprising, after the step C, the step of conducting a dehydration process through a warm or cool dryer to produce Mountain Litsea fruits that can be short-term or long-term stored using refrigerator or freezer.

5. The method according to claim 1, further comprising, after the step C, the steps of baking the superior fruits at a low temperature between 40 and 90° C.; placing the baked fruits in a crusher with pure eater; and crushing the braked fruits into a pulp.

6. The method according to claim 1, further comprising, after the step C, the steps of baking the superior fruits at a low temperature between 40 and 90° C.; placing the baked fruits in a presser; and pressing the baked fruits to produce oil; conducting a powdering process on the oil residue from the pressing process; and conducting a high-temperature, high-pressure process on the oil residue from the pressing process into a green fuel product.

7. The method according to claim 1, further comprising, after the step C, the steps of mixing the superior fruits with pure water and conducting a distillation process at 100° C.; separating oil and water after distillation through an oil-water separation device where the separated water is a pure dew.

8. The method according to claim 7, further comprising the step of separating the oil by a centrifuge into an upper-layered oil, a middle-layered oil, and a bottom-layered collagen.

9. The method according to claim 1, further comprising the step of placing and keeping the product M in a vacuum tank for 1 to 3 years, and then distilling the product M to obtain Mountain Litsea wine; wherein the residual water left from the distillation becomes Mountain Litsea mofetil alcohol anhydride fermented.

10. The method according to claim 9, further comprising the step of removing and drying the yeast left in the vacuum tank into Mountain Litsea yeast
